(19) United States
(12) Patent Application Publication
(10) Pub. No.: US 2025/0213885 A1
(43) Pub. Date: Jul. 3, 2025

(54) DEVICES, SYSTEMS, AND METHODS FOR TREATING ABDOMINAL AORTIC ANEURYSMS

(71) Applicant: Boston Scientific Scimed, Inc., Maple Grove, MN (US)

(72) Inventors: Olena Pernatiy, Galway (IE); Kaylah O'Neill, Roscam (IE); Aiden Flanagan, Galway (IE)

(73) Assignee: Boston Scientific Scimed, Inc., Maple Grove, MN (US)

(21) Appl. No.: 19/008,889

(22) Filed: Jan. 3, 2025

Related U.S. Application Data
(60) Provisional application No. 63/617,233, filed on Jan. 3, 2024.

(51) Int. Cl. A61N 5/06; A61B 17/12; A61M 25/09; A61M 25/10

(52) U.S. Cl. CPC A61N 5/062; A61M 25/1011; A61B 17/12045; A61B 17/12109; A61B 17/12136; A61M 25/09; A61M 2025/1052; A61N 2005/0602; A61N 2005/0651; A61N 2005/0663

(57) ABSTRACT

Devices, systems, and method for treating abdominal aortic aneurysms are disclosed. An illustrative system can include an occlusion catheter with an elongate catheter shaft. The elongate catheter shaft can have a first expandable member and a second expandable member. The elongate catheter shaft can have a therapeutic fluid infusion port disposed in an isolated area between the first expandable member and the second expandable member, where the occlusion catheter is configured to infuse a therapeutic fluid via the elongate catheter shaft and through the therapeutic fluid infusion port to a position proximate to an abdominal aortic aneurysm in the isolated area. The illustrative system including an elongate light-emitting catheter including a light-emitting element, wherein the light-emitting element is configured to emit light and be disposed at the position.

### CROSS-REFERENCE TO RELATED APPLICATIONS

This application claims the benefit of priority under 35 U.S.C. § 119 of U.S. Provisional Application No. 63/617,233, filed Jan. 3, 2024, the entire disclosure of which is hereby incorporated by reference.

### FIELD

This disclosure relates generally to devices, systems, and methods for treating abdominal aortic aneurysms.

### BACKGROUND

Aneurysms are degenerative diseases characterized by destruction of arterial architecture and subsequent dilatation of the blood vessel that may eventually lead to fatal ruptures. Some common locations for aneurysms include the abdominal aorta (abdominal aortic aneurysm, AAA), thoracic aorta, and brain arteries. In addition, peripheral aneurysms of the leg, namely the popliteal and femoral arteries are prevalent locations of this vascular pathology. The occurrence of such peripheral aneurysms appears to be strongly associated with the presence of aneurysms in other locations, as it has been estimated that 30 to 60% of peripheral aneurysm patients also have an AAA.

Aneurysms may grow over a period of time and may pose great risks to health. For instance, aneurysms have the potential to dissect or rupture, causing massive bleeding, stroke, and hemorrhagic shock, which can be fatal in more than 80% of cases. AAAs are a serious health concern, specifically for the aging population, being among the top ten causes of death for patients older than 50. The estimated incidence for abdominal aortic aneurysm is about 50 in every 100,000 persons, with about 200,000 newly diagnosed AAAs annually in the United States alone.

Aneurysms can be caused by any of a large class of degenerative diseases and pathologies including atherosclerotic disease, defects in arterial components, genetic susceptibilities, and high blood pressure, among others, and can develop silently over a period of years. The hallmarks of aneurysms include enzymatic degradation of vascular structural proteins such as elastin, inflammatory infiltrates, calcification, and eventual overall destruction of the vascular architecture. Techniques have been developed to track the progress of the aneurysm using a laboratory test, such as a blood test, a urine test, and/or high resolution imaging technology (CT, MRI) and/or contrast agents or a combination thereof may be used for diagnosing and identifying the degree of expansion of abdominal aortic aneurysms.

Conventional methods of treatment for diagnosed aneurysms are generally limited to invasive surgical techniques. After initial diagnosis of a small aneurysm, the most common medical approach is to maintain active surveillance (e.g., conduct periodic follow-ups) of the development of the aneurysm. The surveillance may continue until the aneurysm such as an AAA reaches a predetermined size. Upon reaching a pre-determined size (e.g., about 5.4 cm in diameter), surgical treatment of the AAA is performed. Current surgical treatments generally are limited to either an endovascular stent graft repair or complete replacement of the diseased vessel with a vascular graft. While such surgical treatments can save lives and improve quality of life for those suffering aneurysm, dangers beyond those of the surgery itself still exist for the patient due to possible post-surgery complications (e.g., neurological injuries, bleeding, or stroke) as well as device-related complications (e.g., thrombosis, leakage, or failure).

Additionally, such surgical interventions are generally not suitable for small (e.g., about 3.0 to 4.4 cm in diameter) and/or medium (e.g., about 4.5 to 5.4 cm in diameter) AAAs. Yet, a large percentage of small and medium sized AAAs progress to become large AAAs which require surgery and/or may have a higher risk of rupture resulting in fatal hemorrhaging in a short amount of time. Further, depending upon the location or anatomy of the aneurysm, the danger of an invasive surgical procedure may outweigh the possible benefits of the procedure, for instance in the case of an aneurysm deep in the brain, leaving the sufferer with very little in the way of treatment options. Moreover, surgical treatments may not always provide a permanent solution, as vascular grafts can loosen and dislodge should the aneurysm progress following the corrective surgery. Generally, most of the current treatment options for aneurysm are mechanical bridges. Yet, for some patients the particular nature of the aneurysm or the condition of the patient makes the patient unsuitable for graft repair and/or mechanical bridges.

It is with these considerations in mind that the improvements of the present disclosure may be useful.

### SUMMARY

This summary of the disclosure is given to aid understanding, and one of skill in the art will understand that each of the various aspects and features of the disclosure may advantageously be used separately in some instances, or in combination with other aspects and features of the disclosure in other instances. No limitation as to the scope of the claimed subject matter is intended by either the inclusion or non-inclusion of elements, components, or the like in this summary. Accordingly, while the disclosure is presented in terms of aspects or embodiments, it should be appreciated that individual aspects can be claimed separately or in combination with aspects and features of that embodiment or any other embodiment.

In some aspects, a system for treating an abdominal aortic aneurysm is provided that comprises an occlusion catheter including: an elongate catheter shaft having: a first expandable member and a second expandable member; and a therapeutic fluid infusion port configured to be disposed in an isolated area between the first expandable member and the second expandable member; wherein the occlusion catheter is configured to infuse a therapeutic fluid via the elongate catheter shaft and through the therapeutic fluid infusion port to a position proximate to an abdominal aortic aneurysm in the isolated area; and an elongate light-emitting catheter including a light-emitting element, wherein the light-emitting element is configured to emit light and be disposed at the position.

In some embodiments, the light-emitting element is configured to emit light having a wavelength in a range from about 495 nanometers to about 570 nanometers.

In some embodiments, which may be used in conjunction with any of the above aspects and embodiments, the light-emitting element is configured to emit the light radially about at least a portion of the elongate light-emitting catheter.

In some embodiments, which may be used in conjunction with any of the above aspects and embodiments, a fluid extraction port is formed in the elongate catheter shaft.

In some embodiments, which may be used in conjunction with any of the above aspects and embodiments, the fluid extraction port is located in the elongate catheter shaft between the first expandable member and the second expandable member.

In some embodiments, which may be used in conjunction with any of the above aspects and embodiments, a perfusion port is formed in the elongate catheter shaft, wherein the perfusion port is disposed outside the isolated area and proximate to the first expandable member or the second expandable member.

In some embodiments, which may be used in conjunction with any of the above aspects and embodiments, a first perfusion port and a second perfusion port are formed in the elongate catheter shaft, wherein the first perfusion port and the second perfusion port are disposed outside the isolated area, wherein the first perfusion port is proximate to the first expandable member, and wherein the second perfusion port is proximate to the second expandable member.

In some embodiments, which may be used in conjunction with any of the above aspects and embodiments, the first expandable member and the second expandable member are expandable balloons.

In some embodiments, which may be used in conjunction with any of the above aspects and embodiments, the therapeutic fluid is a Rose-Bengal solution.

In some embodiments, which may be used in conjunction with any of the above aspects and embodiments, the therapeutic fluid includes a saline solution and a Rose-Bengal solution.

In some embodiments, which may be used in conjunction with any of the above aspects and embodiments, the elongate catheter shaft further comprises a third expandable member.

In some embodiments, which may be used in conjunction with any of the above aspects and embodiments, the light-emitting element includes a light-emitting diode (LED) element or fiber-optic element that is coupled to a light source that is external to the occlusion catheter.

In some embodiments, which may be used in conjunction with any of the above aspects and embodiments, a guide wire extends through the elongate catheter shaft.

In some embodiments, which may be used in conjunction with any of the above aspects and embodiments, the system further comprises a first guide wire and a second guide wire, wherein the first guide wire extends through a main portion and a first portion of the elongate catheter shaft, and wherein the second guide wire extends through the main portion and a second portion of the elongate catheter shaft.

In some embodiments, which may be used in conjunction with any of the above aspects and embodiments, the system further comprises an ultrasonic transducer, wherein the ultrasonic transducer is coupled to the elongate catheter shaft, the elongate light-emitting catheter, or both.

In some aspects, a system for treating an abdominal aortic aneurysm is provided, the system comprising: an occlusion catheter including: an elongate catheter shaft having: a first expandable member and a second expandable member; and a therapeutic fluid infusion port disposed in an isolated area between the first expandable member and the second expandable member; a therapeutic fluid infusion port formed in the elongate catheter shaft, the therapeutic fluid infusion port disposed in the isolated area; a fluid extraction port formed in the elongate catheter shaft, the fluid extraction port disposed in the isolated area, and wherein the occlusion catheter is configured to infuse a first therapeutic fluid and a second therapeutic fluid via the elongate catheter shaft and through the therapeutic fluid infusion port to a position proximate to an abdominal aortic aneurysm; and an elongate light-emitting catheter including a light-emitting element, wherein the light-emitting element is configured to emit light and be disposed at the position.

In some embodiments, the first therapeutic fluid is a saline solution, and the second therapeutic fluid is a Rose-Bengal solution.

In some aspects, a method of treating an abdominal aortic aneurysm is provided, the method comprising: isolating, via an expandable member coupled to an elongate catheter shaft of an occlusion catheter, an area of a lumen that includes an abdominal aortic aneurysm; infusing, via a fluid infusion port of the occlusion catheter, a therapeutic fluid into the area of the lumen proximate to the abdominal aortic aneurysm; and emitting light, via a light-emitting element of an elongate light-emitting catheter, to the isolated area of the lumen proximate to the abdominal aortic aneurysm for a therapeutic amount of time.

In some embodiments, the method further comprises emitting ultrasonic waves to the isolated area of the lumen that includes the abdominal aortic aneurysm.

In some embodiments, which may be used in conjunction with any of the above aspects and embodiments, the therapeutic fluid includes a Rose-Bengal solution.

These and other features and advantages of the present disclosure will be readily apparent from the following detailed description, the scope of the claimed invention being set out in the appended claims.

### BRIEF DESCRIPTION OF THE DRAWINGS

The accompanying drawings, which are incorporated in and constitute a part of this specification, illustrate various exemplary embodiments and together with the description serve to explain the principles of the present disclosure.

Non-limiting embodiments of the present disclosure are described by way of example with reference to the accompanying drawings, which are schematic and not intended to be drawn to scale. The accompanying drawings are provided for purposes of illustration only, and the dimensions, positions, order, and relative sizes reflected in the figures in the drawings may vary. For example, devices may be enlarged so that detail is discernable, but is intended to be scaled down in relation to, e.g., fit within a working channel of a delivery catheter or endoscope. In the figures, identical or nearly identical or equivalent elements are typically represented by the same reference characters, and similar elements are typically designated with similar reference numbers differing in increments of 1000, with redundant description omitted. For purposes of clarity and simplicity, not every element is labeled in every figure, nor is every element of each embodiment shown where illustration is not necessary to allow those of ordinary skill in the art to understand the disclosure.

The detailed description will be better understood in conjunction with the accompanying drawings, wherein like reference characters represent like elements, as follows:

FIG. 1 is a schematic view of a portion of a patient's arterial system.

FIG. 2A is an enlarged schematic view showing a portion of the arterial system of a patient who has been treated via an illustrative system for treating an abdominal aortic aneurysm at a first location.

FIG. 2B is an enlarged schematic view showing a portion of another illustrative occlusion catheter in the illustrative system for treating an abdominal aortic aneurysm.

FIG. 3 is an enlarged schematic view showing a portion of the arterial system of a patient who has been treated via an illustrative system for treating an abdominal aortic aneurysm at a second location.

FIG. 4 is view of an illustrative occlusion catheter for treating an abdominal aortic aneurysm.

FIG. 5 is a schematic illustration of a method of treating an abdominal aortic aneurysm, in accordance with an embodiment of the present disclosure.

While the disclosure is amenable to various modifications and alternative forms, specifics thereof have been shown by way of example in the drawings and will be described in detail. It should be understood, however, that the intention is not to limit the disclosure to the particular embodiments described. On the contrary, the intention is to cover all modifications, equivalents, and alternatives falling within the spirit and scope of the disclosure.

### DETAILED DESCRIPTION

This disclosure is now described with reference to an exemplary medical system that may be used in renal medical procedures. However, it should be noted that reference to this particular procedure is provided only for convenience and not intended to limit the disclosure. A person of ordinary skill in the art would recognize that the concepts underlying the disclosed devices and related methods of use may be utilized in any suitable procedure, medical or otherwise. This disclosure may be understood with reference to the following description and the appended drawings, the same or similar reference numbers will be used through the drawings to refer to the same or like parts.

The term “distal” refers to a portion farthest away from a user when introducing a device into a patient. By contrast, the term “proximal” refers to a portion closest to the user when placing the device into the patient. As used herein, the terms “comprises,” “comprising,” or any other variation thereof, are intended to cover a non-exclusive inclusion, such that a process, method, article, or apparatus that comprises a list of elements does not necessarily include only those elements, but may include other elements not expressly listed or inherent to such process, method, article, or apparatus. The term “exemplary” is used in the sense of “example,” rather than “ideal.” Further, as used herein, the terms “about,” “approximately” and “substantially” indicate a range of values within +/−10% of a stated or implied value. Additionally, terms that indicate the geometric shape of a component/surface refer to exact and approximate shapes.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to affect such feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

Disclosed herein are the systems, devices, and methods herein that may be employed to isolate an area of a lumen that includes an aneurysm such as an abdominal aortic aneurysm, infuse a therapeutic fluid (e.g., a photo activated dye such as a Rose-Bengal solution) into the isolated area, and emit light in the isolated area in the presence of the infused therapeutic fluid for a therapeutic amount of time (e.g., 2 minutes). The therapeutic amount of time may be sufficient to improve vessel distensibility. For instance, such treatment of the isolated area may stimulate growth of new tissue (e.g., new tissue in an aortic wall) in the isolated area.

The growth of the new tissue can affect vessel distensibility, namely may result in the stiffening (e.g., reduction) of distensibility of the vessel in the isolated area. As such, the approaches herein can mitigate the growth of small (e.g., about 3.0 to 4.4 centimeter (cm) in diameter) and/or medium (e.g., about 4.5 to 5.4 cm in diameter) abdominal aortic aneurysms. Such mitigation can reduce a percentage of small and medium sized abdominal aortic aneurysms that subsequently progress to become large abdominal aortic aneurysms which require surgery and/or reduce a percentage of small and/or medium sized abdominal aortic aneurysms that rupture. Yet, such approaches are minimally invasive as compared to traditional surgical interventions such as those typically associated with large diameter abdominal aortic aneurysms.

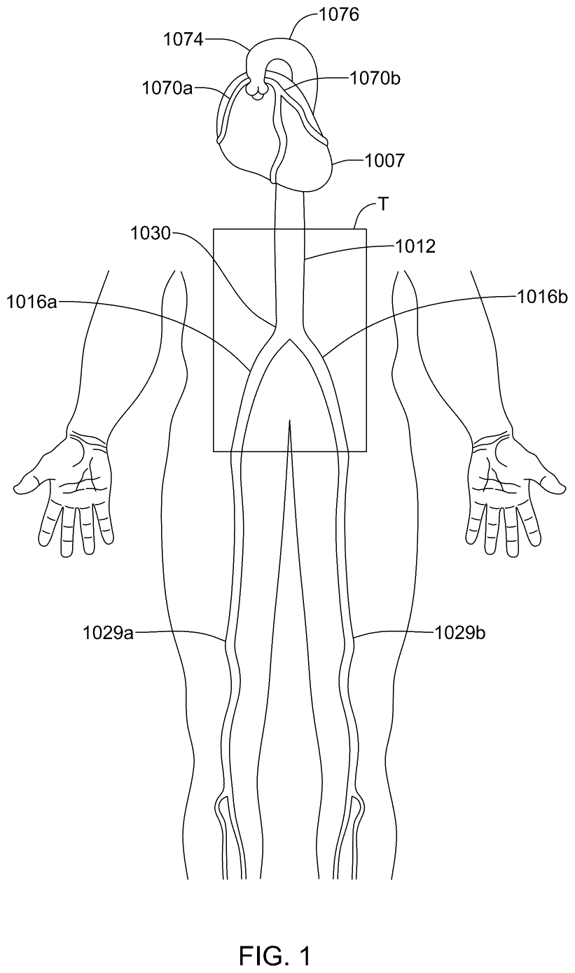

FIG. 1 is a schematic view of a portion of the patient's arterial system. The arterial system of FIG. 1 includes a heart 1007 that pumps blood and an arterial system that distributes oxygen rich blood throughout the body. During each heartbeat, the left ventricle of heart 1007 contracts, pumping blood through the aortic valve and into the ascending aorta 1074. Blood from the ascending aorta 1074 flows through the aortic arch 1076 and down the descending aorta 1012 to the lower body. Blood from the ascending aorta 1074 also flows into the left coronary artery 1070b and the right coronary artery 1070a. In a healthy heart, the left coronary artery 1070b and the right coronary artery 1070a provide a continuous flow of blood to the heart which assures that the heart muscle remains well oxygenated. The descending aorta 1012 continues to the iliac bifurcation 1030, which is a branch that splits into the right common iliac artery 1016a and the left common iliac artery 1016b. The right common iliac artery 1016a becomes the right femoral artery 1029a and the left common iliac artery 1016b becomes the left femoral artery 1029b.

In the exemplary embodiment of FIG. 1, an abdominal aortic aneurysm (not illustrated in FIG. 1) is present in an arterial wall of a blood vessel within a target region T of the patient's arterial system. As blood flows through the aorta, the weakened area corresponding to the abdominal aortic aneurysms may bulge like a balloon and may eventually burst (referred to as rupturing).

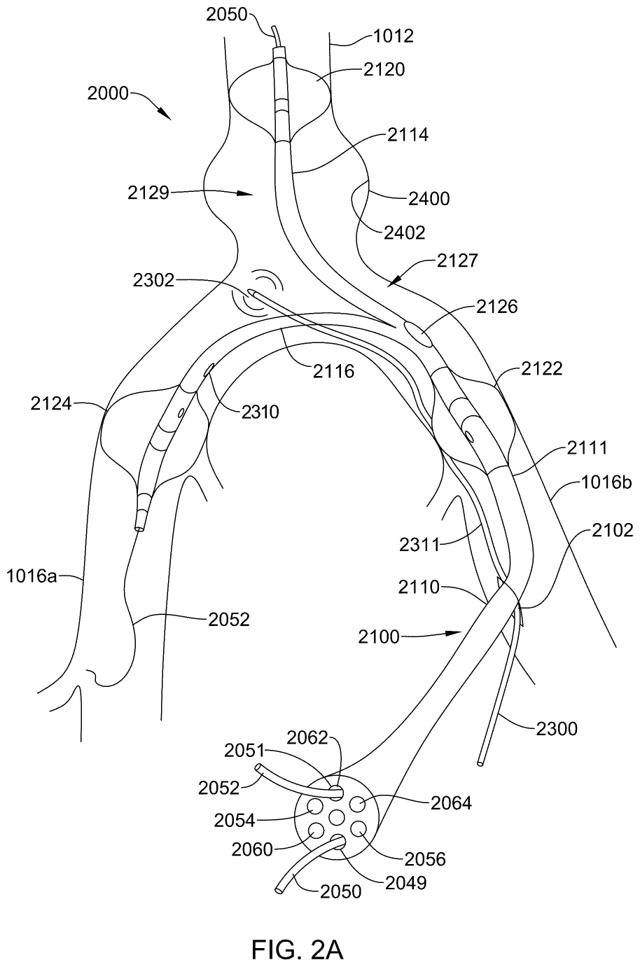

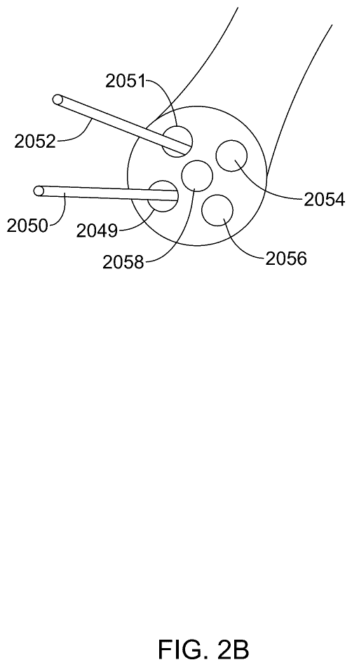

With reference to FIG. 2A, an illustrative system 2000 including an illustrative occlusion catheter 2100 and illustrative elongate light-emitting catheter 2300 is depicted. The illustrative occlusion catheter 2100 and illustrative elongate light-emitting catheter 2300 may be inserted into a patient and advanced to a target area T, as described with respect to FIG. 1. As described herein, the illustrative system 2000 may isolate an area (e.g., isolated section) of an interior of a blood vessel and may subsequently deliver a therapeutic fluid to the isolated area using less invasive procedures than other approaches such as those that employ a full surgical intervention.

The illustrative occlusion catheter 2100 may comprise an elongate catheter shaft 2110 that is inserted into a patient. For instance, as depicted in FIG. 2A the elongate catheter shaft 2110 may be extend through a common iliac artery such as the left common iliac artery 1016b. For example, the elongate catheter shaft 2110 of the occlusion catheter 2100 may be inserted into the femoral artery and extend through the left common iliac artery 1016b, among other possibilities.

The elongate light-emitting catheter 2300 may comprise an elongate catheter shaft 2311 that is inserted into a patient. For instance, as depicted in FIG. 2A the elongate catheter shaft 2311 may be extend through a common iliac artery such as the left common iliac artery 1016b. For example, the elongate catheter shaft 2311 of the elongate light-emitting catheter may be inserted into the femoral artery and extend through the left common iliac artery 1016b, among other possibilities.

In some embodiments, each of the occlusion catheter 2100 and illustrative elongate light-emitting catheter 2300 may be inserted at the same (e.g., a common) insertion point 2102. For instance, both the occlusion catheter 2100 and illustrative elongate light-emitting catheter 2300 may be inserted into an insertion point in the femoral artery and extend through the left common iliac artery 1016b, among other possibilities.

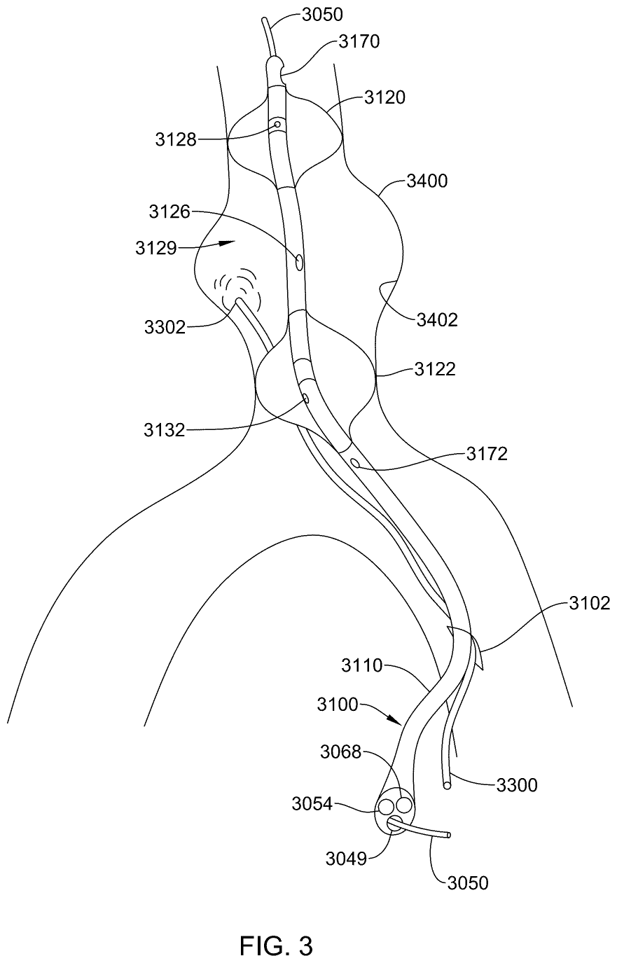

The elongate catheter shaft 2110 may include a first portion 2114 and a second portion 2116. However, in some embodiments, the elongate catheter shaft 2110 may be formed of a unitary member, for instance, as described with respect to FIG. 3. As illustrated in FIG. 2A, the first portion 2114 and the second portion 2116 may extend from (e.g., branch off of) the same point of the main portion 2111 of the elongate catheter shaft 2110. In some embodiments, the first portion 2114 can be configured to extend into descending aorta 1012. In some embodiments, the second portion can be configured to extend into a common iliac artery (e.g., the common iliac artery 1016a) that is proximate to the other common iliac artery (e.g., the common iliac artery 1016b) in which a main portion 2111 of the elongate catheter shaft 2110 is inserted.

In some embodiments, the elongate catheter shaft 2110 may include a guide wire extending through the elongate catheter shaft 2110. For instance, each of the first portion 2114 and the second portion 2116 can include respective guide wires extending therethrough. For example, a first guide wire 2050 can extend through and out of a distal end of the first portion 2114 and a second guide wire 2052 can extend through and out of a distal end of the second portion 2116. The guide wires 2050, 2052 can extend through respective lumens in the main portion 2111 of the elongate catheter shaft 2110, as described with respect to FIG. 2A and FIG. 2B. In some embodiments, the first guide wire 2050 may extend through the main portion 2111 and the first portion 2114 while the second guide wire 2052 may extend through the main portion 2111 and the second portion 2116 of the elongate catheter shaft 2110, as illustrated in FIG. 2A. Employing a guide wire such as the first guide wire 2050 and the second guide wire 2052 may promote positioning of the elongate catheter shaft 2110 within the target area T (e.g., positioning the first portion 2114 in a particular location in the descending aorta 1012 and/or positioning the second portion 2116 at a particular location in a common iliac artery)

The elongate catheter shaft 2110 may include a plurality of expandable members coupled thereto. As detailed herein, the expandable members may be used to seal a portion of a blood vessel, for example, to isolate an entire blood vessel region that has aneurysm or showing signs of aneurysm, such as a section of the aorta below the renal arteries. For instance, the expandable members can be coupled to respective locations on a surface such as an exterior surface of the elongate catheter shaft and can expand radially (e.g., relative to the main, first, and/or second portions of the elongate catheter 2100) to isolate an interior section of a blood vessel that is located between the expandable members. The expandable members may be respective sealing balloons or other types of respective expandable members that are configured to isolate an interior section of a blood vessel.

The expandable members generally have a low-profile delivery configuration (e.g., an uninflated configuration) and a deployed, extended configuration (e.g., an inflated configuration) that contacts and seals against the wall of the vessel. For instance, in some embodiments each of the plurality of expandable members is a respective scaling balloon, as illustrated in FIG. 2A. As illustrated in FIG. 2A, each of the expandable members is in an expanded state, thereby forming an isolated area 2129 between the expandable members (e.g., the first expandable member 2120, the second expandable member 2122, and the third expandable member 2124). Thus, in some embodiments the elongate catheter shaft 2110 may include three expandable members coupled thereto. That is, the elongate catheter shaft 2110 may include only (e.g., exactly) three expandable members coupled thereto in some embodiments. However, in some embodiments fewer or a greater quantity of expandable members may be employed. For instance as described with respect to FIG. 3, in some embodiments the elongate catheter shaft 2110 may include two expandable members coupled thereto. That is, the elongate catheter shaft 2110 may include only (e.g., exactly) two expandable members coupled thereto in some embodiments.

As illustrated in FIG. 2A, the isolated area 2129 may include an aneurysm such as an abdominal aortic aneurysm (AAA) 2400. Accordingly, the systems and methods herein can provide treatments to inhibit and/or reverse the progression of aneurysm, prevent further weakening and dilation of the inner wall 2402 of the AAA 2400.

For instance, a therapeutic fluid infusion port 2126 may be disposed in the isolated area 2129 and the occlusion catheter 2100 may be configured to infuse a therapeutic fluid (represented as element 2127) via the elongate catheter shaft 2110 and through the therapeutic fluid infusion port 2126 to the inner wall 2402 of the AAA 2400 in the isolated area 2129, as detailed herein. As detailed herein, the therapeutic fluid 2127 may include a Rose-Bengal solution. In some embodiments, the therapeutic fluid 2127 may include a saline solution and a Rose-Bengal solution.

In some embodiments, the occlusion catheter 2100 may be configured to infuse only a Rose-Bengal solution (e.g., in the absence of other fluids) via the elongate catheter shaft such as the elongate catheter shaft 2110 and through the therapeutic fluid infusion port 2126 to a position proximate or adjacent to the inner wall 2402 of the abdominal aortic aneurysm 2400 in the isolated area 2129. However, in some embodiments, the occlusion catheter 2100 may be configured to infuse Rose-Bengal solution and an additional fluid prior to, during, or after infusion of the Rose-Bengal solution via the elongate catheter shaft such as the elongate catheter shaft 2110 and through the therapeutic fluid infusion port 2126 to a position proximate or adjacent to the inner wall 2402 of the abdominal aortic aneurysm 2400 in the isolated area 2129. For instance, the occlusion catheter 2100 may be configured to infuse a first therapeutic fluid (e.g., a saline solution) and subsequently infuse as second therapeutic fluid (e.g., a Rose-Bengal solution) that is different than the first therapeutic fluid via the elongate catheter shaft 2110 such as the elongate catheter shaft 2110 and through the therapeutic fluid infusion port 2126 to a position proximate or adjacent to the inner wall 2402 of the abdominal aortic aneurysm 2400 in the isolated area 2129, as described herein.

In some embodiments, the elongate catheter shaft 2110 may include an aspiration or extraction port 2310. For instance, the elongate catheter shaft 2110 may include the extraction port 2310 disposed in the isolated area 2129, as illustrated in FIG. 2A. The extraction port 2310 may be in fluidic communication with a respective lumen in the elongate catheter shaft 2110. The extraction port 2310 can be configured to permit aspirating fluid, via the fluid extraction port 2310 prior to and during infusing (e.g., application) of the therapeutic fluid to mitigate any dilution (e.g., dilution with blood or another fluid) of the therapeutic fluid in the isolated area 2129 of the lumen and/or to reduce pressure within the isolated area 2129 during infusion of the therapeutic fluid.

The elongate light-emitting catheter 2300 may include a light-emitting element 2302. The light-emitting element 2302 may be configured to emit light and be disposed at the position in proximity or adjacent to the inner wall 2402 of the AAA. For instance, the light-emitting element 2302 may be in direct contact with the inner wall 2402 or may be spaced a distance away from the inner wall 2402. In some instances, the light-emitting element may be configured to emit light in the isolated area in the presence of the infused therapeutic fluid for a therapeutic amount of time (e.g., 2 minutes), as described herein. In some embodiments, the light-emitting element 2302 may be configured to emit “green” light having a wavelength in a range from about 495 nanometers to about 570 nanometers. In some embodiments, the light-emitting element 2302 may be configured to emit light radially (e.g., in two dimensions or in three-dimensions) about at least a portion of the elongate light-emitting catheter 2300. For instance, the light-emitting element 2302 may be configured to emit light radially along a 360 degree arc extending about at least a portion of the elongate light-emitting catheter 2300 at which the light-emitting element 2302 is disposed. For instance, in some embodiments, the light-emitting element 2302 may be disposed at a distal tip of the elongate light-emitting catheter 2300, as illustrated in FIG. 2A. In some embodiments, the light-emitting element 2302 may be configured to emit light at least radially about the distal tip of the elongate light-emitting catheter 2300. Having the light-emitting element 2302 be configured to emit light radially promote aspects herein such as causing a photo-activation of the therapeutic fluid which is present at (e.g., in contact with) the inner wall 2402 (e.g., inner vessel wall) of the AAA 2400 in the isolated area 2129, as detailed herein. In some embodiments, the light-emitting element 2302 may be a light-emitting diode (LED) element or may be fiber-optic element that is coupled to a light source that is external to the elongate light-emitting catheter 2300. For instance, the light-emitting element 2302 may be an internal light-emitting diode (LED) element that is coupled to the distal end (e.g., which is implanted in the patient) of the elongate light-emitting catheter 2300. However, in some embodiments, the light-emitting element can be a fiber-optic element or other type of element that is coupled to an external laser or other type of light source that is external to the patient.

In some embodiments, the system 2000 can further include an ultrasonic transducer that is configured to emit ultrasonic waves. For instance, the system 2000 can include an ultrasonic transducer (not shown) coupled to the elongate catheter shaft 2110, the elongate light-emitting catheter 2300, or both. That is, the ultrasonic transducer can be configured to emit ultrasonic waves and to be disposed internally within that patient at the position proximate or adjacent to the abdominal aortic aneurysm. However, in some embodiments, the system 2000 can further include an ultrasonic transducer that is separate from the elongate catheter shaft 2110 and the elongate light-emitting catheter 2300. For instance, the ultrasonic transducer may be located external to the patient in some instances. It is contemplated that providing ultrasonic waves from an ultrasonic transducer in combination with the light from the elongate light-emitting catheter 2300 may promote aspects herein such as promoting enhanced diffusion of the therapeutic fluid into a vessel wall (e.g., in an aneurysm) and/or promoting enhanced cross-linking (e.g., quick and/or a greater degree of collagen cross-linking), as compared to other approaches that may expose a Rose-Bengal solution to light or ultrasonic waves alone.

As illustrated in FIG. 2A, the occlusion catheter 2100 may include a plurality of lumens extending therethrough. The lumens may provide flow passages between locations at or near ports such as an infusion port to locations at or near the proximal end of the occlusion catheter 2100. The occlusion catheter 2100 may include a total of three or more, four or more, five or more, six or more, or seven or more lumens, among other possibilities. For instance, the occlusion catheter 2100 may include a total of seven lumens, as illustrated in FIG. 2A, among other possibilities. For example, the occlusion catheter 2100 may include a total of five lumens, as illustrated in FIG. 2B.

The occlusion catheter 2100 may include a guide wire lumen such as a first guide wire lumen 2049 and a second guide wire lumen 2051. The first guide wire lumen 2049 and the second guide wire lumen may each be configured to have a respective guide wire disposed therethrough. For instance, the first guide wire lumen 2049 may be configured to have the first guide wire 2050 disposed therethrough and the second guide wire lumen 2051 may be configured to have the second guide wire 2052 disposed therethrough.

The occlusion catheter 2100 may include an extraction lumen 2056. The extraction lumen 2056 may be coupled to an extraction port such as the extraction port 2310. As such, the extraction lumen 2056 in conjunction with the extraction port may permit aspirating fluid prior to and during infusing (e.g., application) of the therapeutic fluid to mitigate any dilution (e.g., dilution with blood or another fluid) of the therapeutic fluid in the isolated area 2129 of the lumen and/or to reduce pressure within the isolated area 2129 during infusion of the therapeutic fluid. The occlusion catheter 2100 may include an infusion lumen 2054. The infusion lumen 2054 may be configured to permit infusion of a therapeutic fluid to be delivered to the therapeutic fluid infusion port 2126 such as those described herein. In some embodiments, the infusion lumen 2054 can extend through the occlusion catheter 2100.

The occlusion catheter 2100 may include an inflation lumen such as a first inflation lumen 2060, a second inflation lumen 2062, and a third inflation lumen 2064. The first inflation lumen 2060 can correspond to the first expandable member 2120, the second inflation lumen 2062 can correspond to the second expandable member 2122, and the third inflation lumen 2064 can correspond to the third expandable member 2124. Each respective inflation lumen can be configured to selectively expand or deflate a corresponding expandable member. That is, as illustrated in FIG. 2A, the occlusion catheter 2100 can include a respective (e.g., individual) inflation lumen for each expandable member. In such embodiments, the individual inflation lumens can be inflated at the same or different times to form the isolated area 2129. For instance, in some embodiments, each of the individual inflation lumens can be inflated at the same time to form the isolated area 2129.

However, in some embodiments an inflation lumen can correspond to a plurality of expandable members. For instance, as illustrated in FIG. 2B, an individual inflation lumen 2058 can correspond to each of the expandable member (e.g., the first expandable member 2120, the second expandable member 2122, and the third expandable member 2124) included in the occlusion catheter 2100. That is, FIG. 2B is an enlarged schematic view showing a portion of another illustrative occlusion catheter in the illustrative system for treating an abdominal aortic aneurysm in which an individual expansion lumen is employed.

FIG. 3 is an enlarged schematic view showing a portion of the arterial system of a patient who has been treated via an illustrative system for treating an abdominal aortic aneurysm at a second location. The system 3000 in FIG. 3 is analogous to the system 2000 in FIG. 2A but with the change that the occlusion catheter 2100 is formed of a unitary elongate catheter shaft 3110 with a first expandable member 3120, a second expandable member 3122, a first perfusion port 3170 and a second perfusion port 3172.

The illustrative occlusion catheter 3100 may comprise an elongate catheter shaft 3110 that is inserted into a patient, for instance based on a guidewire 3050. For instance, as depicted in FIG. 3 the elongate catheter shaft 3110 may be extend through a common iliac artery such as the left common iliac artery 1016b. For example, the elongate catheter shaft 3110 of the occlusion catheter 3100 may be inserted into the femoral artery and extend through the right common iliac artery 3016b, among other possibilities.

In some embodiments, each of the occlusion catheter 3100 and illustrative elongate light-emitting catheter 3300 may be inserted at the same (e.g., a common) insertion point 3102. For instance, both the occlusion catheter 3100 and illustrative elongate light-emitting catheter 3300 may be inserted into an insertion point in the femoral artery and extend through the left common iliac artery 1016b, among other possibilities.

The elongate catheter shaft 3110 may be formed of a unitary member in the form of the main portion 3111, as illustrated in FIG. 3. As such, each of the expandable members may extend (e.g., extend radially) from the unitary member that forms the elongate catheter shaft 3110. For instance, a first expandable member 3120 and a second expandable member 3122 may be coupled to the elongate catheter shaft 3110 and may expand radially from the elongate catheter shaft to form an isolated area 3129. For instance, the first expandable member 3120 may be coupled to a lumen in the elongate catheter shaft 3110 and may be configured to radially expand responsive to the introduction of fluid via the lumen and an orifice 3128 into the first expandable member 3120. Similarly, the second expandable member 3122 may be coupled to a lumen in the elongate catheter shaft 3110 and may be configured to expand radially from responsive to the introduction of fluid via the lumen and an orifice 3132 into the second expandable member.

As mentioned, a therapeutic fluid infusion port 3126 may be disposed in the isolated area 3129. In some embodiments, the occlusion catheter 3100 may be configured to infuse a Rose-Bengal solution via the elongate catheter shaft 3110 and through the infusion lumen 3056 and the therapeutic fluid infusion port 3126 to a position proximate or adjacent to the inner wall 3402 of the abdominal aortic aneurysm 3400 in the isolated area 3129. In some embodiments, the occlusion catheter 3100 may be configured to infuse the Rose-Bengal solution and an additional fluid prior to, during, or after infusion of the Rose-Bengal solution via the infusion lumen 3054 along the elongate catheter shaft 3110 through the therapeutic fluid infusion port 3126 to a position proximate or adjacent to the inner wall 3402 of the abdominal aortic aneurysm 3400 in the isolated area 3129. For instance, in some embodiments, the occlusion catheter 3100 may be configured to infuse a first therapeutic fluid (e.g., a saline solution) and subsequently infuse as second therapeutic fluid (e.g., a Rose-Bengal solution) that is different than the first therapeutic fluid via the elongate catheter shaft such as the elongate catheter shaft 3110 and through the therapeutic fluid infusion port 3126 to a position proximate or adjacent to the inner wall 3402 of the abdominal aortic aneurysm 3400 in the isolated area 3129, as described herein. In some embodiments, the elongate light-emitting catheter 3300 may include a light-emitting element 3302 that is configured to emit light and be disposed at the position proximate or adjacent to the inner wall 3402 of the AAA 3400. In some embodiments, the light-emitting catheter 3300 and thus the light-emitting element 3302 may be disposed at the position proximate or adjacent to the inner wall 3402 of the AAA 3400 and may remain static during emission of the light to the inner wall 3402. However, in some instances, the light-emitting catheter 3300 and the light-emitting element 3302 may be disposed at the position proximate or adjacent to the inner wall 3402 of the AAA 3400 and my be moved (e.g.,. rotated, translated, etc.) relative the inner wall 3402 during emission of the light.

In some embodiments, the elongate catheter shaft 3110 may include a perfusion port. The perfusion portion may be disposed outside the isolated area 3129. For instance, the elongate catheter shaft 2110 may include a perfusion port disposed outside the isolated area 3131 and proximate to the first expandable member 3120 or the second expandable member 3122. For example, the elongate catheter shaft 3110 may include a first perfusion port 3170 and a second perfusion port 3172 formed in the elongate catheter shaft 3110. The perfusion port refer to orifices or openings extending radially through a surface of the elongate catheter shaft 3110 into one or more lumens in the elongate catheter shaft 3110. The first perfusion port 3170 and/or the second perfusion port 3172 may be located outside the isolated area 3129. As illustrated in FIG. 3, each of the first perfusion port 3170 and the second perfusion port 3172 may be disposed outside the isolated area 3129. For instance, the first perfusion port 3170 may be proximate to the first expandable member 3120 and disposed outside of the isolated area 3131 and the second perfusion port 3172 may be proximate to the second expandable member 2122 and be disposed outside of the isolated area 3131. The perfusion portion may permit the flow of liquid (e.g., blood and/or therapeutic fluid) into or around the isolated area 3129. For instance, the first perfusion port 3170 and the second perfusion portion 3172 may be fluidically coupled to permit the flow of blood around the isolated area 3129 thereby minimizing an amount of blood in the isolated area (e.g., to minimize diffusion of the therapeutic fluid in the isolated area) and/or to reduce the pressure in the isolated area 3129. For instance, the first perfusion port 3170 and the second perfusion portion 3172 may be fluidically coupled to permit the flow of blood around the isolated area when the first expandable member 3120 and the second expandable member 3122 are inflated.

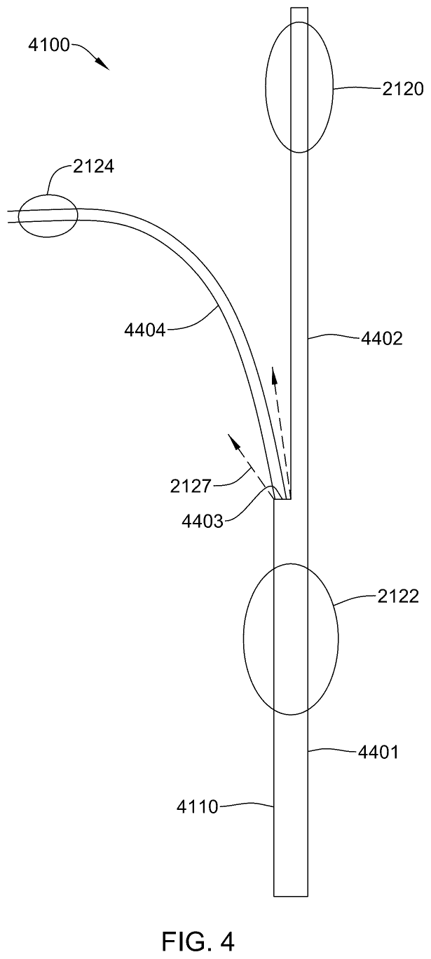

FIG. 4 is view of an illustrative occlusion catheter 4100 for treating an abdominal aortic aneurysm. The occlusion catheter 4100 is similar to the occlusion catheter 2100 with the change that a first portion 4402 is a linear extension of a main portion 4401 of the occlusion catheter 4100 and a second portion 4404 extends from a lumen included in the main portion 4401 of the occlusion catheter 4100. That is, as illustrated in FIG. 4, the first portion 4402 may be a extend along a common plane as the main portion 4401 of the occlusion catheter 4100 while the second portion 4404 may extend at an angle to the common plane.

In some embodiments, the second portion 4404 may extend out of a lumen that forms or is proximate or adjacent to a lumen forming an injection and/or an extraction lumen. For instance, the second portion 4404 may extend out of a lumen the forms an injection lumen and that is configured to inject the therapeutic fluid out of a lumen subsequent to extension of the second portion 4404 a distance out of the lumen. Stated differently, in some embodiments, a lumen extending through the main portion 4401 can be configured to provide a dual function of permitting an expandable member such as a third expandable member 2124 to expand out of an expansion port 4403 that is located in the main portion 4401 of an elongate catheter shaft 4110 and subsequently injecting the therapeutic fluid out of the same lumen and the same expansion port 4403.

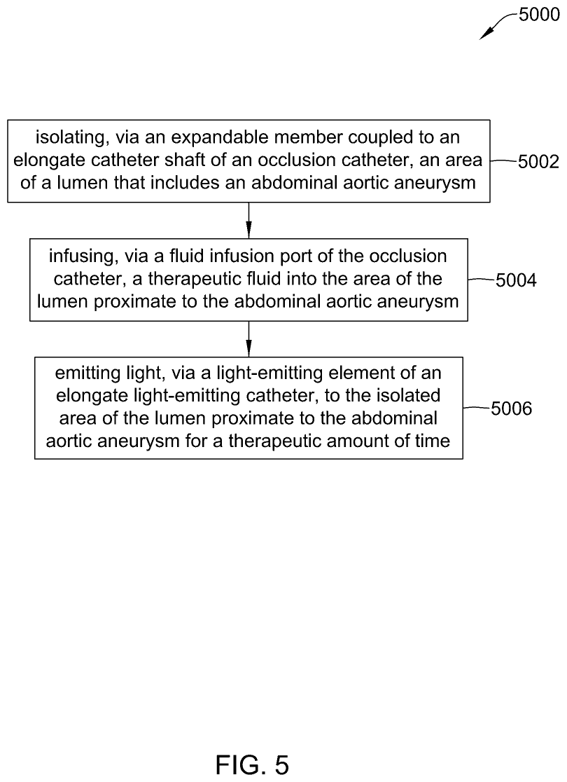

FIG. 5 is a schematic illustration of a method 5000 of treating an abdominal aortic aneurysm, in accordance with an embodiment of the present disclosure. At 5002, the method 5000 can include isolating an area of a lumen that includes an abdominal aortic aneurysm. For instance, the method 5000 can include isolating, via an expandable member coupled to an elongate catheter shaft of an occasional catheter, an area of a lumen that includes an abdominal aortic aneurysm, as described herein. In some embodiments, isolating the area can include employing two or more expandable members to mitigate or cease fluidic flow (e.g., blood flow) into and/or out of an area of a lumen that includes an abdominal aortic aneurysm.

In some embodiments, the isolation of the lumen (e.g., blood vessel wall) can be performed with two or more expandable members that provide for continued blood flow past the isolated region based on a specific conduit or an appropriately designed opening through the structure providing the isolated treatment location including the abdominal aortic aneurysm. For instance, as described herein a perfusion port or a plurality of perfusion ports can be located proximal or distal to the isolated treatment area to permit fluid flow (e.g., blood flow) past the isolated treatment area.

At 5004, the method 5000 can include infusing a therapeutic fluid into the area of the lumen proximate (e.g., within the isolated area) or adjacent to the abdominal aortic aneurysm. In some embodiments, the method 5000 can include infusing, via a fluid infusion port of a catheter, a therapeutic fluid into the area of the lumen proximate or adjacent to the abdominal aortic aneurysm. For instance, the method 5000 can include infusing, via the fluid infusion port of the occlusion catheter, the therapeutic solution into the area of the lumen proximate or adjacent to the abdominal aortic aneurysm subsequent to isolating the area, as described at 5002. For example, an occlusion catheter can include an infusion port located between two or more expandable members that is configured to infuse a therapeutic fluid into the area of the lumen proximate or adjacent to the abdominal aortic aneurysm when each of the two or more expandable members is expanded. That is, as mentioned the occlusion catheter herein can include an infusion port on the occlusion catheter that is configured to infuse a therapeutic fluid into the area of the lumen proximate or adjacent to the abdominal aortic aneurysm.

In some embodiments, the method 5000 can include infusing, via the fluid infusion port of the occlusion catheter, a Rose-Bengal solution into the area of the lumen proximate or adjacent to the abdominal aortic aneurysm. For instance, in some embodiments, the therapeutic fluid includes only (consists of) a Rose-Bengal solution (e.g., including Rose-Bengal component and a suitable solvent or diluent in addition to the Rose-Bengal component). However, in some embodiments an additional therapeutic fluid such as a saline solution can be employed. In such embodiments, the therapeutic fluids may include only (consist of) a saline solution and a Rose-Bengal solution. While the therapeutic solution is generally described in terms of include a Rose-Bengal solution the disclosure is not so limited. Rather, other suitable photo-activated solutions or stains such as other xanthene stains which include a xanthene ring may be employed.

At 5006, the method 5000 can include emitting light, via a light-emitting element of an elongate light-emitting catheter, to the area of the lumen proximate (e.g., within the isolated area along a linear plane (e.g., a line of sight) from the abdominal aortic aneurysm) or adjacent to the abdominal aortic aneurysm for a therapeutic amount of time. For instance, the method 5000 can include emitting the light in vivo, via the light-emitting element of an elongate light-emitting catheter, to the area of the lumen proximate or adjacent to the abdominal aortic aneurysm for the therapeutic amount of time subsequent to infusing the therapeutic fluid into the area (e.g., the isolated area) of the lumen proximate or adjacent to the abdominal aortic aneurysm. Emission of the light may activate some or all of the therapeutic fluid (e.g., a photo-activated dye such as a Rose-Bengal solution) in the area (e.g., in the isolated area) of the lumen. As mentioned, the light-emitting element can be located on (e.g., on an end of) an elongate light-emitting catheter that is separate from the elongate catheter shaft. However, it is contemplated that in some embodiments, the light-emitting element can be included (e.g., on an outer housing and/or extend through a lumen thereof) of the elongate catheter shaft.

In general, a therapeutic fluid comprises Rose-Bengal in a suitable solvent or diluent. A therapeutic fluid can optionally comprise additional agents or carriers. Such additional agent can be active agents, providing direct benefit to the tissue, or may be supporting agents, improving delivery, compatibility, or reactivity of other agents in the composition. For example, in one embodiment, the compositions can be combined with any of a number of possible lipid-lowering medications so as to prevent the development of calcified lipid deposits or arteriosclerosis plaques that can often be found in conjunction with aneurysm formation. Lipid lowering medications may include, for example, statins, niacin and fibrates. In some embodiments, the therapeutic fluid can comprise one or more buffers. For example, a composition may be formulated with inclusion of purified water, saline and a biocompatible buffer, such as phosphate buffers, borate buffers, HEPES, PIPES, and MOPSO. In some embodiments, a composition may be formulated to have a pH of between about 5.5 and about 7.4. In one embodiment, the composition has a pH of about 6.2. Examples of additional carriers include suitable aqueous and nonaqueous carriers, diluents, solvents or vehicles include water, ethanol, polyols (e.g., glycerol, propylene glycol, polyethylene glycol and the like), carboxymethylcellulose and suitable mixtures thereof; vegetable oils (e.g., olive oil) and injectable organic esters such as ethyl oleate. In addition, the composition can contain minor amounts of auxiliary substances such as wetting or emulsifying agents, pH buffering agents and the like that can enhance the effectiveness of the phenolic compound. These compositions may also contain adjuvants such as preservatives, wetting agents, emulsifying agents, surfactants and dispersing agents. In some embodiments, the therapeutic fluid may include various antibacterial and antifungal agents such as paraben, chlorobutanol, phenol, sorbic acid and the like. It may also be desirable to include isotonic agents such as sugars, sodium chloride and the like in the therapeutic fluid.

The concentration of a Rose-Bengal of the therapeutic fluid can be in the range of about 0.0001% to about 25% (w/v). In some embodiments, the concentration can be in the range of about 0.005% to about 10% (w/v). In some embodiments, the concentration can be in the range of about 0.01% to about 1% (w/v). A person of ordinary skill in the art will recognize that additional ranges of concentration within the explicit ranges above are contemplated and are within the present disclosure.

In some embodiments, infusing, via the fluid infusion port of the occlusion catheter, can introduce the therapeutic fluid into the area of the lumen proximate to or adjacent to the abdominal aortic aneurysm for a total duration of time between about 1 minute and about 5 minutes. Stated differently, the therapeutic amount of time for the in vivo infusing, via the fluid infusion port of the occlusion catheter, can introduce the therapeutic fluid into the area of the lumen proximate or adjacent to the abdominal aortic aneurysm for a total duration of time between about 1 minute and about 5 minutes. For instance, infusing via the fluid infusion port of the occlusion catheter, can introduce the therapeutic fluid continuously into the area of the lumen proximate or adjacent to the abdominal aortic aneurysm for a total duration of time between about 1 minute and about 5 minutes. In some instances, a light-emitting element can emit light in the isolated area in the presence of the infused therapeutic fluid for a therapeutic amount of time (e.g., 2 minutes) that is less than, equal to, or exceeds, the amount of time that the therapeutic fluid is introduced into the area of the lumen proximate or adjacent to the abdominal aortic aneurysm. For instance, the light-emitting element can emit light in the isolated area in the presence of the infused therapeutic fluid for a therapeutic amount of time (e.g., 2 minutes) that equal to or exceeds, the amount of time that the therapeutic fluid is introduced into the area of the lumen proximate to or adjacent to the abdominal aortic aneurysm. Having the light-emitting element can emit light in the isolated area in the presence of the infused therapeutic fluid for a therapeutic amount of time (e.g., 2 minutes) that equal to or exceeds, the amount of time that the therapeutic fluid is introduced into the area can promote aspects herein such as stimulating growth of new tissue (e.g., new tissue in an aortic wall) in the isolated area.

It is contemplated that providing and activating (e.g., photo-activating) the Rose-Bengal solution proximate (e.g., into the isolated area of the lumen) or adjacent to the abdominal aortic aneurysm can decrease vessel distensibility due at least to facilitating the cross-linking of collagen present in lumen adjacent to the abdominal aortic aneurysm. Yet, the approaches herein notably do not require invasive surgery and do not require the presence of additional foreign materials such as stents, tissue solder materials and/or a tissue bonding materials. Accordingly, the approaches herein can mitigate the time and/or quantity of foreign materials employed within the patient's body and yet result in the stiffening (e.g., reduction in distensibility) of the vessel in the isolated area thereby mitigating the growth of small and/or medium abdominal aortic aneurysms. As mentioned, such mitigation can reduce a percentage of small and medium abdominal aortic aneurysms that subsequently progress to become large abdominal aortic aneurysms which require surgery and/or reduce a percentage of small and/or medium sized abdominal aortic aneurysms that rupture.

As mentioned, in some embodiments the therapeutic fluid can include a plurality of therapeutic fluids such as a first therapeutic fluid and a second therapeutic fluid. In some embodiments, that first therapeutic fluid can be infused into the isolated area for a first period of time and the second therapeutic fluid can be infused into the isolated area for a second period of time that is subsequent to the first period of time. For example, a first therapeutic fluid may be a saline solution that is infused for into the isolated area (e.g., via an infusion port of an occlusion catheter) for a first period of time and the second therapeutic fluid may be a Rose-Bengal solution that is infused into the isolated area (e.g., via the infusion port of an occlusion catheter) for a second period of time that is subsequent to the first period of time. In some embodiments, the first therapeutic solution (e.g., a saline solution) and the second therapeutic solution (e.g., a Rose-Bengal solution) may be infused at different times via the same infusion port included in an occlusion catheter. The first period of time can be the same or different than the second period of time. For instance, the first period of time (e.g., 1 min) can be less the second period of time (e.g., 2 min). However, in some embodiments, the first period of time (e.g., 3 minutes) can be greater than the second period of time (e.g., 2 minutes). In some embodiments, each of the first period of time and the second period of time can have a total duration of time between about 1 minute and about 5 minutes.

In some embodiments, the method 5000 can include aspirating the area of the lumen that includes an abdominal aortic aneurysm. For instance, the method 5000 can include aspirating the isolated area of the lumen that includes an abdominal aortic aneurysm prior to, during, and/or following infusing of the therapeutic fluid. For example, the method 5000 can include aspirating the isolated area of the lumen that includes an abdominal aortic aneurysm prior to and during infusing (e.g., application) of the therapeutic fluid to mitigate any dilution (e.g., dilution with blood or another fluid) of the therapeutic fluid in the isolated area of the lumen and/or to reduce pressure within the isolated area. For instance, the occlusion catheters herein may include a dedicated extraction lumen and/or another lumen such as those disclosed above may be repurposed to provide aspiration of the area of the lumen that includes an abdominal aortic aneurysm prior to, during, and/or following infusion of the therapeutic fluid. Alternatively, or in addition, a separate catheter (separate from the occlusion catheter) may include a lumen that is employed to provide aspiration of the area of the lumen that includes an abdominal aortic aneurysm prior to, during, and/or following application of the therapeutic solution.

In some embodiments, the method 5000 may include emitting ultrasonic waves to the isolated area of the lumen that includes the abdominal aortic aneurysm. For instance, the method 5000 may include emitting ultrasonic waves at the same time as emitting light from the elongate light-emitting catheter 2300 to promote aspects herein such as promoting enhanced diffusion of the therapeutic fluid as detailed above, as compared to other approaches that may expose a Rose-Bengal solution to light or ultrasonic waves alone.

It will be apparent to those skilled in the art that various modifications and variations can be made to the disclosed device without departing from the scope of the disclosure. Other embodiments of the disclosure will be apparent to those skilled in the art from consideration of the specification and practice of the disclosure disclosed herein. It is intended that the specification and examples be considered as exemplary only, with a true scope and spirit of the disclosure being indicated by the following claims.

All apparatuses and methods discussed herein are examples of apparatuses and/or methods implemented in accordance with one or more principles of this disclosure. These examples are not the only way to implement these principles but are merely examples. Thus, references to elements or structures or features in the drawings must be appreciated as references to examples of embodiments of the disclosure, and should not be understood as limiting the disclosure to the specific elements, structures, or features illustrated. Other examples of manners of implementing the disclosed principles will occur to a person of ordinary skill in the art upon reading this disclosure.

In the foregoing description and the following claims, the following will be appreciated. The phrases “at least one”, “one or more”, and “and/or”, as used herein, are open-ended expressions that are both conjunctive and disjunctive in operation. The term “a” or “an” entity, as used herein, refers to one or more of that entity. As such, the terms “a” (or “an”), “one or more” and “at least one” can be used interchangeably herein. All directional references (e.g., proximal, distal, upper, lower, upward, downward, left, right, lateral, longitudinal, front, back, top, bottom, above, below, vertical, horizontal, radial, axial, clockwise, counterclockwise, and/or the like) are only used for identification purposes to aid the reader's understanding of the present disclosure, and/or serve to distinguish regions of the associated elements from one another, and do not limit the associated element, particularly as to the position, orientation, or use of this disclosure. Connection references (e.g., attached, coupled, connected, and joined) are to be construed broadly and may include intermediate members between a collection of elements and relative movement between elements unless otherwise indicated. As such, connection references do not necessarily infer that two elements are directly connected and in fixed relation to each other. Identification references (e.g., primary, secondary, first, second, third, fourth, etc.) are not intended to connote importance or priority, but are used to distinguish one feature from another.

The foregoing discussion has been presented for purposes of illustration and description and is not intended to limit the disclosure to the form or forms disclosed herein. It will be understood that various additions, modifications, and substitutions may be made to embodiments disclosed herein without departing from the concept, spirit, and scope of the present disclosure. In particular, it will be clear to those skilled in the art that principles of the present disclosure may be embodied in other forms, structures, arrangements, proportions, and with other elements, materials, and components, without departing from the concept, spirit, or scope, or characteristics thereof. For example, various features of the disclosure are grouped together in one or more aspects, embodiments, or configurations for the purpose of streamlining the disclosure.

However, it should be understood that various features of the certain aspects, embodiments, or configurations of the disclosure may be combined in alternate aspects, embodiments, or configurations. One skilled in the art will appreciate that the disclosure may be used with many modifications of structure, arrangement, proportions, materials, components, and otherwise, used in the practice of the disclosure, which are particularly adapted to specific environments and operative requirements without departing from the principles of the present disclosure. For example, elements shown as integrally formed may be constructed of multiple parts or elements shown as multiple parts may be integrally formed, the operation of elements may be reversed or otherwise varied, the size or dimensions of the elements may be varied, and features and components of various embodiments may be selectively combined. The presently disclosed embodiments are therefore to be considered in all respects as illustrative and not restrictive, the scope of the claimed invention being indicated by the appended claims, and not limited to the foregoing description.

The following claims are hereby incorporated into this Detailed Description by this reference, with each claim standing on its own as a separate embodiment of the present disclosure. In the claims, the term “comprises/comprising” does not exclude the presence of other elements or steps. Furthermore, although individually listed, a plurality of means, elements or method steps may be implemented by, e.g., a single unit or processor. Additionally, although individual features may be included in different claims, these may possibly advantageously be combined, and the inclusion in different claims does not imply that a combination of features is not feasible and/or advantageous. In addition, singular references do not exclude a plurality. The terms “a”, “an”, “first”, “second”, etc., do not preclude a plurality. Reference signs in the claims are provided merely as a clarifying example and shall not be construed as limiting the scope of the claims in any way.

What is claimed is:

1. A system for treating an abdominal aortic aneurysm, the system comprising:
an occlusion catheter including:
an elongate catheter shaft having:
a first expandable member and a second expandable member; and
a therapeutic fluid infusion port configured to be disposed in an isolated area between the first expandable member and the second expandable member;
wherein the occlusion catheter is configured to infuse a therapeutic fluid via the elongate catheter shaft and through the therapeutic fluid infusion port to a position proximate to an abdominal aortic aneurysm; and
an elongate light-emitting catheter including a light-emitting element, wherein the light-emitting element is configured to emit light and be disposed at the position.

2. The system of claim 1, wherein the light-emitting element is configured to emit light having a wavelength in a range from about 495 nanometers to about 570 nanometers.

3. The system of claim 2, wherein the light-emitting element is configured to emit the light radially about at least a portion of the elongate light-emitting catheter.

4. The system of claim 1, further comprising a fluid extraction port formed in the elongate catheter shaft.

5. The system of claim 4, wherein the fluid extraction port is located in the elongate catheter shaft between the first expandable member and the second expandable member.

6. The system of claim 5, further comprising a perfusion port formed in the elongate catheter shaft, the perfusion port disposed outside the isolated area and proximate to the first expandable member or the second expandable member.

7. The system of claim 6, further comprising a first perfusion port and a second perfusion port formed in the elongate catheter shaft, the first perfusion port and the second perfusion port disposed outside the isolated area, the first perfusion port proximate to the first expandable member, and the second perfusion port proximate to the second expandable member.

8. The system of claim 1, wherein the first expandable member and the second expandable member are expandable balloons.

9. The system of claim 1, wherein the therapeutic fluid is a Rose-Bengal solution.

10. The system of claim 1, wherein the therapeutic fluid includes a saline solution and a Rose-Bengal solution.

11. The system of claim 1, wherein the elongate catheter shaft further comprises a third expandable member.

12. The system of claim 1, wherein the light-emitting element includes a light-emitting diode (LED) element or fiber-optic element that is coupled to a light source that is external to the occlusion catheter.

13. The system of claim 1, further comprising a guide wire extending through the elongate catheter shaft.

14. The system of claim 1, further comprising a first guide wire and a second guide wire, wherein the first guide wire extends through a main portion and a first portion of the elongate catheter shaft, and wherein the second guide wire extends through the main portion and a second portion of the elongate catheter shaft.

15. The system of claim 1, further comprising an ultrasonic transducer, wherein the ultrasonic transducer is coupled to the elongate catheter shaft, the elongate light-emitting catheter, or both.

16. A system for treating an abdominal aortic aneurysm, the system comprising:
an occlusion catheter including:
an elongate catheter shaft having:
a first expandable member and a second expandable member; and
a therapeutic fluid infusion port disposed in an isolated area between the first expandable member and the second expandable member;
a therapeutic fluid infusion port formed in the elongate catheter shaft, the therapeutic fluid infusion port disposed in the isolated area;
a fluid extraction port formed in the elongate catheter shaft, the fluid extraction port disposed in the isolated area, and
wherein the occlusion catheter is configured to infuse a first therapeutic fluid and a second therapeutic fluid via the elongate catheter shaft and through the therapeutic fluid infusion port to a position proximate to an abdominal aortic aneurysm; and
an elongate light-emitting catheter including a light-emitting element, wherein the light-emitting element is configured to emit light and be disposed at the position proximate to the abdominal aortic aneurysm responsive.

17. The system of claim 16, wherein:
the first therapeutic fluid is a saline solution; and
the second therapeutic fluid is a Rose-Bengal solution.

18. A method of treating an abdominal aortic aneurysm, the method comprising:
isolating, via an expandable member coupled to an elongate catheter shaft of an occlusion catheter, an area of a lumen that includes an abdominal aortic aneurysm;
infusing, via a fluid infusion port of the occlusion catheter, a therapeutic fluid into the area of the lumen proximate to the abdominal aortic aneurysm; and
emitting light, via a light-emitting element of an elongate light-emitting catheter, to the isolated area of the lumen proximate to the abdominal aortic aneurysm for a therapeutic amount of time.

19. The method of claim 18, further comprising emitting ultrasonic waves to the isolated area of the lumen that includes the abdominal aortic aneurysm.

20. The method of claim 19, wherein the therapeutic fluid includes a Rose-Bengal solution.
